(19) United States
(12) Patent Application Publication
(10) Pub. No.: US 2013/0209542 A1
(43) Pub. Date: Aug. 15, 2013

(54) PHARMACEUTICAL CARRIER AND PHARMACEUTICAL COMPOSITION FOR INHIBITING ANGIOGENESIS

(71) Applicant: National Chiao Tung University, (US)

(72) Inventors: Kuang-Wen LIAO, Hsinchu City (TW); Shu-Yi HO, Kaohsiung City (TW)

(73) Assignee: NATIONAL CHIAO TUNG UNIVERSITY, Hsinchu City (TW)

(21) Appl. No.: 13/680,388

(22) Filed: Nov. 19, 2012

(30) Foreign Application Priority Data
Feb. 15, 2012 (TW) 101104952

(51) Int. Cl. A61K 47/42; A61K 9/127; A61K 9/48

(52) U.S. Cl. CPC A61K 47/42; A61K 9/4891; A61K 9/127
USPC 424/450; 424/400; 424/491

(57) ABSTRACT

A pharmaceutical carrier and a pharmaceutical composition for inhibiting angiogenesis are disclosed. The pharmaceutical carrier of the present invention comprises: a drug carrier; and a polypeptide linked to a surface of the drug carrier, wherein the polypeptide comprises a receptor binding domain of vascular endothelial growth factor.

### CROSS REFERENCE TO RELATED APPLICATION

This application claims the benefits of the Taiwan Patent Application Serial Number 101104952, filed on Feb. 15, 2011, the subject matter of which is incorporated herein by reference.

### BACKGROUND OF THE INVENTION

1. Field of the Invention

The present invention relates to a pharmaceutical carrier and a pharmaceutical composition for inhibiting angiogenesis and, more particularly, to a pharmaceutical carrier and a pharmaceutical composition capable of targeting tumor cells and inhibiting angiogenesis.

2. Description of Related Art

Angiogenesis is one common physiological process, which can be found in wound healing, female menstrual period and fetal growth.

However, angiogenesis is also one important factor related to tumor growth. After tumor development, tumor cells or surrounding tissues may secrete several materials capable of inducing angiogenesis, and tumor cells also obtain nutrients through angiogenic blood vessels. In addition, tumor cells may distribute to circulatory system through angiogenic blood vessels, and new blood vessels are formed on other organs to develop metastasis cancer. Hence, the metastasis of tumor cells is also highly related to angiogenesis.

Since the tumor development is highly related to angiogenesis, several therapies are developed to inhibit angiogenesis, in order to inhibit tumor growth. For example, monoclonal antibody, Avastin, is one effective targeting drug for inhibiting angiogenesis. However, this drug may be metabolized or distribute to undesired organs.

Except for directly inhibiting angiogenesis, some studies desire to inhibit the function or the generation of vascular endothelial growth factors, since vascular endothelial growth factors are known as materials inducing angiogenesis. When the function or the generation of vascular endothelial growth factors is inhibited, the purpose of inhibiting angiogenesis can be accomplished, and the purpose of inhibiting tumor growth can further be obtained.

Therefore, it is desirable to provide a drug or a method directed to vascular endothelial growth factors, and especially a targeting drug or a method using the same, in order to accomplish the purpose of inhibiting tumor growth and treating cancer.

### SUMMARY OF THE INVENTION

An object of the present invention is to provide a pharmaceutical carrier for inhibiting angiogenesis, which can recognize vascular endothelial growth factor receptor (VEGFR) to obtain a purpose of releasing drugs in specific positions.

Another object of the present invention is to provide a pharmaceutical composition for inhibiting angiogenesis, which has a capability to target vascular endothelial growth factor receptor to obtain a purpose of treating diseases related to angiogenesis.

To achieve the object, the pharmaceutical carrier for inhibiting angiogenesis of the present invention comprises: a drug carrier; and a polypeptide linked to a surface of the drug carrier, wherein the polypeptide comprises a receptor binding domain of vascular endothelial growth factor (RBDV).

In addition, the pharmaceutical composition for inhibiting angiogenesis of the present invention comprises: a pharmaceutical carrier; and an active ingredient. Herein, the pharmaceutical carrier comprises: a drug carrier; and a polypeptide linked to a surface of the drug carrier, wherein the polypeptide comprises a receptor binding domain of vascular endothelial growth factor. In addition, the active ingredient is encapsulated in the pharmaceutical carrier.

According to the aforementioned pharmaceutical carrier and pharmaceutical composition for inhibiting angiogenesis of the present invention, a polypeptide comprising a receptor binding domain of vascular endothelial growth factor is linked to a surface of the drug carrier. Hence, the pharmaceutical carrier and pharmaceutical composition can target to vascular endothelial growth factor receptor (VEGFR), and especially VEGFR on tumor cells to perform sequential treatments. Preferably, the polypeptide is linked to the surface of the drug carrier by absorption. More preferably, the absorption is accomplished by static electric force.

In the pharmaceutical carrier and pharmaceutical composition for inhibiting angiogenesis of the present invention, the weight ratio of the polypeptide to the drug carrier is 0.002-1.0. Preferably, the weight ratio of the polypeptide to the drug carrier is 0.02-0.6. More preferably, the weight ratio of the polypeptide to the drug carrier is 0.1-0.4, and for example, each 50 μg of drug carrier can carry 5-20 μg of the polypeptide.

In the pharmaceutical composition for inhibiting angiogenesis of the present invention, the active ingredient is an anti-cancer drug or a nucleic acid molecule. Herein, the nucleic acid molecule can be a gene with treating efficacy, such as a nucleotide sequence of a receptor binding domain of vascular endothelial growth factor.

In addition, in the pharmaceutical carrier and pharmaceutical composition for inhibiting angiogenesis of the present invention, the pharmaceutical carrier may further comprise a nucleic acid molecule linked to the surface of the drug carrier, wherein the nucleic acid molecule comprises a nucleotide sequence of a receptor binding domain of vascular endothelial growth factor. Herein, the nucleic acid molecule can be linked to the surface of the drug carrier through chemical bonding or other linking means. Preferably, the nucleic acid molecule is linked to the surface of the drug carrier by absorption. More preferably, the absorption is accomplished by static electric force.

In one aspect that the surface of the drug carrier is combined with a nucleic acid molecular comprising a nucleotide sequence of a receptor binding domain of vascular endothelial growth factor or in another aspect that the active ingredient of the pharmaceutical composition is a nucleic acid molecule comprising a nucleotide sequence of a receptor binding domain of vascular endothelial growth factor, after the pharmaceutical carrier or the pharmaceutical composition targets to the cells through the polypeptide comprising the receptor binding domain of vascular endothelial growth factor, the nucleic acid molecule comprising the nucleotide sequence of the receptor binding domain of vascular endothelial growth factor can enter into the cells and then express proteins or polypeptides corresponding to the nucleic acid molecule inside the cells. The expressed proteins or polypeptides of the receptor binding domain of vascular endothelial growth factor (RBDV) can serve as a competitor to vascular endothelial growth factor (VEGF). The competition between the expressed proteins (or polypeptides) of RBDV and endogeneous VEGF for vascular endothelial growth factor receptor (including receptor 1 and receptor 2, i.e. VEGFR1 and VEGFR2) can accomplish the purpose of inhibiting angiogenesis. It is well known that the growth of tumor cells is highly related to angiogenesis. Hence, the pharmaceutical carrier of the present invention can not only inhibit angiogenesis, but also inhibit tumor cell growth or treat cancers while the pharmaceutical carrier targets to vascular endothelial growth factor receptor of tumor cells.

In one aspect that the surface of the drug carrier is combined with a nucleic acid molecular comprising a nucleotide sequence of a receptor binding domain of vascular endothelial growth factor, the weight ratio of nucleic acid molecule to the drug carrier is 0.01-1.0. Preferably, the weight ratio of nucleic acid molecule to the drug carrier is 0.1-0.6. More preferably, the weight ratio of nucleic acid molecule to the drug carrier is 0.2-0.3, and for example each 50 μg of drug carrier can carry 10-15 μg of the nucleic acid molecule.

In the pharmaceutical carrier and pharmaceutical composition for inhibiting angiogenesis of the present invention, the polypeptide may further comprise a fragment of immunoglobulin, and the fragment of immunoglobulin is linked to the receptor binding domain of vascular endothelial growth factor.

Furthermore, in the pharmaceutical carrier and the pharmaceutical composition for inhibiting angiogenesis of the present invention, the nucleic acid molecule may be a plasmid containing the nucleotide sequence of the receptor binding domain of vascular endothelial growth factor, or a plasmid containing the nucleotide sequence of the receptor binding domain of vascular endothelial growth factor and a nucleotide sequence of a fragment of immunoglobulin. Preferably, the nucleic acid molecule is a plasmid containing the nucleotide sequence of the receptor binding domain of vascular endothelial growth factor and the nucleotide sequence of a fragment of immunoglobulin. More preferably, the nucleic acid molecule is a plasmid that can express the receptor binding domain of vascular endothelial growth factor and the immunoglobulin together to form a fusion protein.

In the pharmaceutical carrier and the pharmaceutical composition for inhibiting angiogenesis of the present invention, preferably, the receptor binding domain of vascular endothelial growth factor is a receptor binding domain of vascular endothelial growth factor A. More preferably, the receptor binding domain of vascular endothelial growth factor is a receptor binding domain of human vascular endothelial growth factor A.

In addition, in the pharmaceutical carrier and the pharmaceutical composition for inhibiting angiogenesis of the present invention, preferably, the aforementioned fragment of immunoglobulin is a constant region fragment of immunoglobulin G1 (IgG). More preferably, the aforementioned fragment of immunoglobulin is a constant region fragment (Fc) of immunoglobulin G1. Most preferably, the aforementioned fragment of immunoglobulin is a constant region fragment of human immunoglobulin G1. The immunoglobulin G1, and especially the constant region fragment thereof has excellent immune properties, so the treatment effect of the pharmaceutical carrier and the pharmaceutical composition can further be improved.

In the pharmaceutical carrier and the pharmaceutical composition of the present invention, the drug carrier may be at least one selected from the group consisting of a liposome, a micelle, a microsphere, a nanoparticle, and a dendrimer. Preferably, the drug carrier is a liposome.

Since the sequence of the receptor binding domain of vascular endothelial growth factor may be variant among different species, one skilled in the art can understand that sequence similarities between sequences of the receptor binding domain of vascular endothelial growth factor and the sequence represented by SEQ ID NO: 1 among different species may be existed when these sequences are analyzed with sequence alignment means such as ClustalW or NCBI BLAST. If some amino acids in the sequence of the receptor binding domain of vascular endothelial growth factor are changed to other amino acids with similar properties such as the exchange between arginine and asparagine and these changes do not influence the interaction between RBDV and VEGFR, the changed amino acids and amino acid sequences are also within the scope of the present invention. Hence, the proteins or polypeptides with 70-100% sequence similarity to the sequence represented by SEQ ID NO: 1 can accomplish the effect of the present invention. Preferably, the receptor binding domain of vascular endothelial growth factor of the present invention has 70-100% sequence identity to the sequence represented by SEQ ID NO: 1. Most preferably, the amino acid sequence of the receptor binding domain of vascular endothelial growth factor of the present invention is represented by SEQ ID NO: 1, and the nucleotide sequence thereof is represented by SEQ ID NO: 2.

In the present invention, the term “similarity” refers to the percentage of similar amino acid residues. Not only identical amino acid residues, but also the amino acid residues with similar properties are defined as similar amino acid residues. Additionally, the term “identity” refers to the percentage of identical amino acid residues or nucleotides.

The pharmaceutical composition of the present invention may further comprise a pharmaceutically acceptable carrier, such as activators, excipients, adjuvants, dispersants, wetting agents, and suspensions.

In the pharmaceutical compositions of the present invention, the term “pharmaceutically acceptable carrier” means that the carrier must be compatible with the active ingredients (and preferably, capable of stabilizing the active ingredients) and not be deleterious to the subject to be treated. In addition, the term “treating” or “treatment” used in the present invention refers to the application or administration of the pharmaceutical compositions of the present invention to a subject with symptoms or tendencies of suffering from cancer in order to cure, heal, alleviate, relieve, alter, remedy, ameliorate, improve, prevent or affect the symptoms or tendencies of angiogenesis or tumor growth.

Furthermore, different angiogenesis disease or cancers can be treated based on the active ingredients encapsulated in the drug carrier. For example, when 5-FU is encapsulated in the drug carrier, the pharmaceutical composition can be used to treat colon cancer.

In addition, the pharmaceutical compositions of the present invention can be administered via parenteral, inhalation, local, rectal, nasal, sublingual, or vaginal delivery, or implanted reservoir. Herein, the term “parenteral delivery” includes subcutaneous, intradermic, intravenous, intra-articular, intra-arterial, synovial, intrapleural, intrathecal, local, and intracranial injections.

Other objects, advantages, and novel features of the invention will become more apparent from the following detailed description when taken in conjunction with the accompanying drawings.

### BRIEF DESCRIPTION OF THE DRAWINGS

FIG. 1 is a result showing a targeting activity of liposome combined with RBDV-IgG1 Fc proteins according to Testing Example 1 of the present invention;

FIG. 2 is a result showing protein expressions of plasmids containing a nucleotide sequence of RBDV-IgG1 Fc in cells according to Testing Example 2 of the present invention;

FIG. 3 is a result showing protein expressions of RBDV-IgG1 Fc proteins in vivo according to Testing Example 5 of the present invention;

FIG. 4 is a result showing inhibition effect of in vivo expressed RBDV-IgG1 Fc proteins on tumor cell growth according to Testing Example 6 of the present invention;

FIG. 5 is a result showing inhibition effect of in vivo expressed RBDV-IgG1 Fc proteins on tumor cell growth according to Testing Example 7 of the present invention; and

FIG. 6 is a graph showing survival rates of mice according to Testing Example 7 of the present invention.

### DETAILED DESCRIPTION OF THE PREFERRED EMBODIMENT

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

### PREPARATIVE EXAMPLE 1

### Preparations of a Plasmid Containing a Nucleotide Sequence of a Receptor Binding Domain of Vascular Endothelial Growth Factor and a Nucleotide Sequence of a Fragment of Immunoglobulin G1 (pAAV-MCS/RBDV-IgG1 Fc), and a Plasmid Containing a Nucleotide Sequence of a Fragment of Immunoglobulin G1 (pAAV-MCS/IgG1 Fc)

Total RNAs were extracted from human epidermoid carcinoma A431, and cDNA fragments of vascular endothelial growth factor (VEGF) were obtained via reverse transcriptase polymerase chain reaction (RT-PCR) by using primers represented by SEQ ID NO: 3 (5′-TGG TGA GAG ATC TGG TTC CCG AAA-3′) and SEQ ID NO: 4 (5′-TTT CGG GAA CCA GAT CTC TCA CCA-3′). The obtained cDNA fragments were used as a template for sequential polymerase chain reaction.

Next, primers containing BamHI restriction site and XhoI restriction site respectively were used in polymerase chain reaction (PCR), wherein the forward primer was represented by SEQ ID NO: 5 (5′-AGG ATC CAT GAA CTT TCT GCT GTC TTG G-3′) and the reverse primer was represented by SEQ ID NO: 6 (5′-ACT CGA GTT AGA TCC GCA TAA TCT GCA TGG T-3′). After PCR, a nucleotide sequence of human receptor binding domain of VEGF (RBDV) was obtained, which corresponded to amino acid residues 1-109 of VEGF protein.

A nucleotide sequence of a constant region fragment (Fc) of immunoglobulin G1 (IgG Fc) was obtained through PCR, wherein pcDNA3.1 expression plasmids containing Fc and IL-2 (Invitrogen, USA) were used as a template, the forward primer was represented by SEQ ID NO: 7 (5′-CGC ATC ATC ACC ATC ACC ATT GAA-3′), and the reverse primer was represented by SEQ ID NO: 8 (5′-AGC TTT CAA TGG TGA TGG TGA TGA TGC GGG CC-3′).

The methods for preparing PCR mixtures to obtain nucleotide sequences of RBDV and IgG Fc are shown as follows. First, 1 μl of the template (50 ng/μl), 1 μg of the forward primer (10 mM), 1 μg of the reverse primer (10 mM), 0.5 μl of Pfu polymerase, 1 μl of dNTP (25 mM) and 5 μl of PCR buffer solution were mixed, and then de-ionized water was added into the mixture to a total volume of 50 μl.

Next, the mixture for PCR was reacted at 94° C. for 30 sec. The primer annealing step was performed at 54° C. for 30 sec, the primer extension step was performed at 72° C. for 2 mM, and the primer annealing step and the extension step were repeated for 34 cycles. Finally, the mixture was reacted at 72° C. for 10 min, and stored at 4° C. to complete PCR.

RBDV fragments obtained from PCR was cut with BamHI and XhoI restriction enzymes and then ligated to N-terminal of IgG Fc. The obtained fused fragment of RBDV and IgG Fc was cut with BamHI and ApaI restriction enzymes, and constructed into a vector of pAAV-MCS (Stratagene, USA), and a His-tag is also constructed into the vector. The forward primer for His-tag was represented by SEQ ID NO: 7 (5′-CGC ATC ATC ACC ATC ACC ATT GAA-3′) and the reverse primer therefor was represented by SEQ ID NO: 8 (5′-AGC TTT CAA TGG TGA TGG TGA TGA TGC GGG CC-3′). Finally, the accuracy of the constructed sequence of pAAV-MCS/RBDV-IgG1 Fc was confirmed by DNA sequencing.

In the following examples, pAAV-MCS/IgG1 Fc construct containing a His-tag was used as a control.

### PREPARATIVE EXAMPLE 2

### Expressions of RBDV-IgG1 Fc and IgG1 Fc Recombinant Proteins

The obtained pAAV-MCS/RBDV-IgG1 Fc and pAAV-MCS/IgG1 Fc constructs were respectively transformed into human embryonic kidney (HEK) 293T cells (obtained from Food Industry Research and development Institute), and cultured in Dulbecco's modified Eagle's medium (DMEM; Invitrogen, Gaithersburg, Md., USA) containing 5% fetal bovine serum qualified (FBS; Invitrogen) and 1% Penicillin-Streptomycin-Amphotericin (PSA; Biological industries, NY, USA) at 37° C. and 5% CO₂ for 48 hr.

After cell lysed, a supernatant was collected, purified with protein G-Agarose (Upstate Inc., Lake Placid, N.Y., USA), and further purified with nickel-charged His-Trap Hp affinity column (Amersham Biosciences, Piscataway, N.J., USA). Finally, Sephadex G-25 prepacked column (Amersham Biosciences, Uppsala, Sweden) was used to change the solution into PBS buffer, and the obtained recombinant proteins were concentrated with Microcon Centrifugal Filter Unit (Millipore, Bedford, Mass., USA).

### PREPARATIVE EXAMPLE 3

### Preparation of Liposome (LPPC)

In the present example, liposomes were synthesized according to Yen-Ku Liu, et al., 2011. A Unique and Potent Protein Binding Nature of Liposome Containing Polyethylenimine and Polyethylene Glycol: A Nondisplaceable Property. Biotechnology and Bioengineering. Briefly, two kinds of lipids and two kinds of polymers were used to prepare liposomes. The lipids were 1,2-Dioleoyl-sn-glycero-3-phosphocholine (DOPC) and 1,2-dilauroyl-sn-glycero-3-phosphocholine (DLPC), and both of them were available from Avanti Polar Lipids (Alabaster, Ala.). The polymers were polyethylene glycol (PEG, MW 15000 and 8000) and Polyetherimide (PEI, MW 25000). The molar ratio of phospholipids:PEG:PEI was about 13:5:5.

### PREPARATIVE EXAMPLE 4

### Preparation of Liposome Labeled with DiO (DiO-LPPC Complex)

100 μl of liposomes obtained from Preparative Example 3 and 10 μl of 2.5 mM DiO solution was mixed and placed for 30 min. Next, 1 ml of de-ionized water was added into the mixture, and the mixture was put into a centrifuge to remove the supernatant. DiO used herein is a fluorescent material.

Then, the precipitant was re-suspended with 100 μl of de-ionized water to obtain liposomes labeled with DiO of the present example (DiO-LPPC complex).

### TESTING EXAMPLE 1

### Evaluation of Targeting Activity of RBDV-IgG1 Fc on DiO-LPPC Complex

50 μg of DiO-LPPC complex was incubated with 0, 0.24, 0.48, 2.4, or 4.8 μg of RBDV-IgG1 Fc or IgG1 Fc for 30 min. The complexes formed by protein and DiO-LPPC (i.e. RBDV-IgG1 Fc-DiO-LPPC complex or IgG1 Fc-DiO-LPPC complex) were introduced into B16/F10 cells, and then the fluorescence intensity of DiO was analyzed with flow cytometer. B16/F10 cells used herein were cells that can express VEGFR-1 and VEGFR-2.

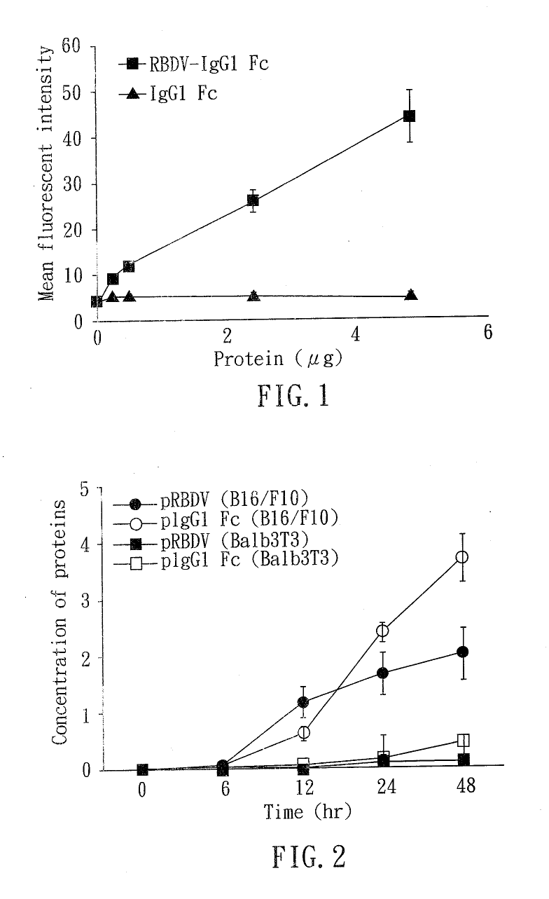

The result is shown in FIG. 1, wherein the X-axis indicates the amount of recombinant proteins, and the Y-axis indicates the fluorescence intensity of DiO. As shown in FIG. 1, as the amount of the recombinant proteins increased, more RBDV-IgG1 Fc-DiO-LPPC complex can bind to cells (as shown in RBDV-IgG1 Fc in FIG. 1) compared to IgG1 Fc-DiO-LPPC complex (as shown in IgG1 Fc in FIG. 1). This result indicates that RBDV-IgG1 Fc-DiO-LPPC complex has targeting ability to cells by the binding of RBDV-IgG1 Fc protein to VEGFR on cells.

### TESTING EXAMPLE 2

### Evaluation of in Vitro Transfection Efficiency of RBDV-IgG1 Fc DNA-LPPC Complex

B16/F10 and Balb3T3 cells were used in the present example, wherein Balb3T3 cells are cells that do not express VEGFR.

B16/F10 and Balb3T3 cells were transfected with pAAV-MCS/RBDV-IgG1 Fc (pRBDV) and pAAV-MCS/IgG1 Fc (pIgG1 Fc). The transfected cells were cultured with the same medium and methods illustrated above. After 48 hr, the cells were analyzed with ELISA at different culturing time points.

The process for performing ELISA is shown as follows. First, a 96-well plate was coated with anti-His-tag antibodies, and placed overnight. Next, the plate was washed with PBST for three times, and water in the plate was removed. Then, the plate was fixed with skim milk powder for 1 hr and washed with PBST for three times, and water in the plate was removed.

Medium containing B16/F10 and Balb3T3 cells transfected with pAAV-MCS/RBDV-IgG1 Fc (pRBDV) and pAAV-MCS/IgG1 Fc (pIgG1 Fc) was added into the 96-well plate and reacted for 1 hr to make anti-His-tag antibodies recognize expressed proteins. The plate was washed with PBST for three times, and then water in the plate was further removed.

Anti-human IgG HRP antibodies were added into the plate and reacted for 1 hr. The plate was washed with PBST for three times, and then water in the plate was further removed. TMB solution was added therein and stained for 20 min, and then 1 N HCl was added therein to stop the reaction. The result was measured with ELISA reader under 450 nm.

FIG. 2 shows an ELISA result, wherein the X-axis indicates the incubation time, and the Y-axis indicates the concentration of expressed proteins. B16/F10 cells transfected with pAAV-MCS/RBDV-IgG1 Fc can steadily express RBDV-IgG1 Fc proteins, but there are no target proteins expressed by Balb3T3 cells. This result indicates that RBDV-IgG1 Fc proteins can be expressed by cells when the plasmid containing RBDV-IgG1 Fe gene was transfected into cells capable of expressing VEGFR.

### PREPARATIVE EXAMPLE 5

### Preparation of Liposome Carrying with Lipophilic Drug (DiI-LPPC Complex)

100 μl of liposomes prepared in Preparative Example 3 were mixed with 10 μl of 10 mM DiI solution and placed for 30 min. Next, 1 ml of de-ionized water was added into the mixture, and the mixture was put into a centrifuge to remove the supernatant. DiI used herein is a fluorescent lipophilic drug.

Then, the precipitant was re-suspended with 100 μl of de-ionized water to obtain liposomes carrying with lipophilic drug (DiI-LPPC complex).

### TESTING EXAMPLE 3

### Evaluation of in Vivo Targeting Activity of RBDV-IgG1 Fc on DiI-LPPC Complex

20 μg of RBDV-IgG1 Fc proteins was incubated with 1 mg of DiI-LPPC complex to obtain complexes of RBDV-IgG1 Fc proteins and liposomes carrying with DiI (RBDV-IgG1 Fc-DiI-LPPC complex). Herein, DiI is a red fluorescent lipophilic drug.

C57/BL6 mice were used in the present testing example. B 16/F10 cells were injected into right flanks of mice, and Balb3T3 cells were injected into left flanks of mice. When the tumor average volume was up to 50 mm³, RBDV-IgG1 Fc-DiI-LPPC complexes were subcutaneously injected into both the right and left flanks. At 0, 48 and 72 hr post-injection, Caliper IVIS system (IVIS Spectrum) was used to observe the in vivo distribution of RBDV-IgG1 Fc-DiI-LPPC complexes in the C57/BL6 mice. The absorption wavelength of DiI is 600 nm, and the emission wavelength thereof is 465 nm.

The result indicates that lipophilic drug DiI can be carried into B16/F10 cells by the complexes of RBDV-IgG1 Fc proteins and liposomes. In addition, the result also indicates that the complexes of RBDV-IgG1 Fc proteins and liposomes only targeted to B16/F10 cells, and did not target to other cells and organs.

Hence, the complexes of RBDV-IgG1 Fc proteins and liposomes have targeting ability to tumor cells, and especially to tumor cells capable of expressing VEGFR. In addition, the complexes of RBDV-IgG1 Fc proteins and liposomes also can be used to carry drugs to target positions. Therefore, when the complexes of RBDV-IgG1 Fc proteins and liposomes are used as pharmaceutical carriers, the RBDV-IgG1 Fc proteins can be used as targeting molecules and the liposomes can be used as drug carriers to carry drugs, so the effect of treating cancers or diseases related to angiogenesis can be accomplished.

### TESTING EXAMPLE 4

### Evaluation of in Vivo Targeting Activity of RBDV-IgG1 Fc on LPPC Complex

100 μg of reporter plasmid DNA was mixed with 1 mg liposomes, and then mixed with 20 μg of RBDV-IgG1 Fc. The reporter plasmid DNA used herein was pAsRed2-N1, which carries red fluorescent proteins under the control of CMV promoter.

C57/BL6 mice were used in the present testing example. B16/F10 cells were injected into right flanks of mice, and Balb3T3 cells were injected into left flanks of mice. When the tumor size reached to 50 mm³, RBDV-IgG1 Fc/LPPC complexes carrying with the reporter plasmid DNA were subcutaneously injected into both the right and left flanks.

At 0, 2, 3 and 6 day post-injections, Caliper IVIS system (IVIS Spectrum) was used to observe the in vivo distribution of RBDV-IgG1 Fc/LPPC complexes in the C57/BL6 mice. The absorption wavelength for observing red fluorescent proteins expressed by pAsRed2-N1 is 600 nm, and the emission wavelength thereof is 465 nm.

The result indicates that RBDV-IgG1 Fc/LPPC complexes only targeted to B16/F10 cells, and did not target to other cells and organs.

Hence, the complexes of RBDV-IgG1 Fc proteins and liposomes have targeting ability to tumor cells and can introduce DNA to target positions. Therefore, when the complexes of RBDV-IgG1 Fc proteins and liposomes are used as pharmaceutical carriers, the complexes can carry DNA and the complexes carrying with DNA can be applied to gene therapies or other therapies for cancers or diseases related to angiogenesis.

### TESTING EXAMPLE 5

### In Vivo Expression of RBDV-IgG1 Fc Protein

Complexes of LPPC and RBDV-IgG1 Fc proteins (LPPC/RBDV protein), complexes of LPPC, pAAV-MCS/IgG1 Fc and RBDV-IgG1 Fc proteins (LPPC/IgG1 plasmid/RBDV protein), complexes of LPPC, pAAV-MCS/RBDV-IgG1 Fc and IgG1 Fc proteins (LPPC/RBDV plasmid/IgG1 protein), and complexes of LPPC, pAAV-MCS/RBDV-IgG1 Fc and RBDV-IgG1 Fc proteins (LPPC/RBDV plasmid/RBDV protein) were used in the present example; and PBS buffer was used as a control. Herein, the mixed ratio of proteins:plasmids:and liposomes was 1 μg:5 μg:50 μg.

LPPC/RBDV protein, LPPC/IgG1 plasmid/RBDV protein, LPPC/RBDV plasmid/IgG1 protein, LPPC/RBDV plasmid/RBDV protein, and PBS buffer were subcutaneously injected into C57/BL6 mice (6-8 weeks of age), and serum thereof was collected at different time points and analyzed with ELISA.

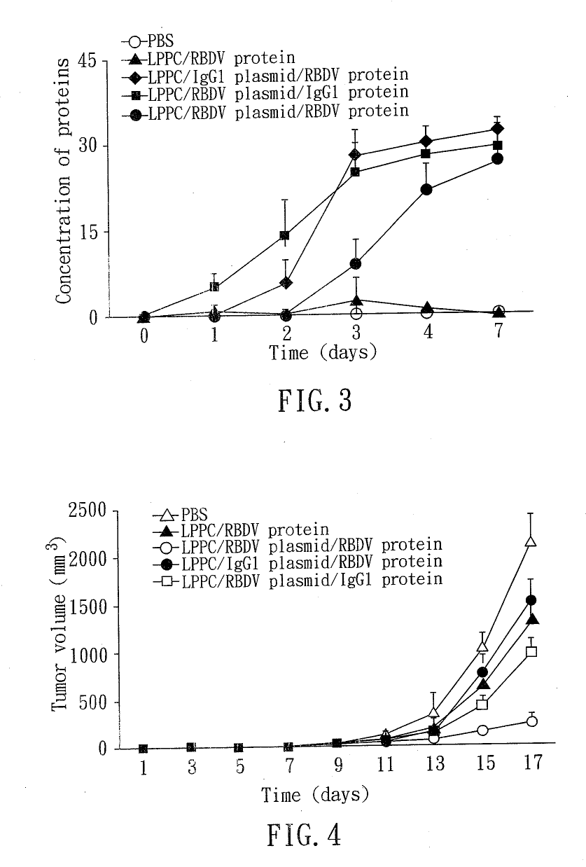

FIG. 3 shows an ELISA result, wherein the X-axis indicates the amount of the proteins, and the Y-axis indicates the fluorescence intensities emitted from DiO. As shown in FIG. 3, the complexes carrying with IgG1 plasmid or RBDV plasmid (i.e. LPPC/IgG1 plasmid/RBDV protein, LPPC/RBDV plasmid/IgG1 protein and LPPC/RBDV plasmid/RBDV protein) can express proteins. Especially, the complexes carrying with RBDV plasmid (i.e. LPPC/RBDV plasmid/IgG1 protein and LPPC/RBDV plasmid/RBDV protein) can steadily express RBDV-IgG1 Fc proteins.

### TESTING EXAMPLE 6

### Inhibiting Ability of in Vivo Expressed RBDV-IgG1 Fc Proteins to Tumor Cells

C57/BL6 mice (6-8 weeks of age) were inoculated with 1×10⁶ cells subcutaneously in 100 ml PBS. When the tumor average volume was up to 30 mm³ (about 9 days post-injection), mice were intravenously (i.v.) injected with the complexes as illustrated in Testing Example 5; and PBS buffer was used as a control.

Then, the tumor volume was measured at different time points, and the result is shown in FIG. 4, wherein the X-axis indicates days of post-injections, and the Y-axis indicates the average volume of tumors. The result shows that the growth of tumor cells can be significantly inhibited when the mice were administered with the complexes containing RBDV plasmids (i.e. LPPC/RBDV plasmid/IgG1 protein and LPPC/RBDV plasmid/RBDV protein). Especially, when the mice were administered with LPPC/RBDV plasmid/RBDV protein, the growth rate of tumor cells was greatly decreased.

This result indicates when liposomes carrying with plasmids containing RBDV gene, the plasmids can express RBDV proteins in vivo and therefore the effect of inhibiting tumor growth can be obtained. In addition, when liposomes further carry with RBDV-IgG1 Fc protein, the effect of inhibiting the tumor growth can further be improved due to the targeting ability of RBDV-IgG1 Fc protein to tumor cells.

### TESTING EXAMPLE 7

### Inhibition of Tumor Growth by in Vivo Expressed RBDV-IgG1 Fc Proteins

The method for performing the present example was the same as that performed in Testing Example 6, except the following differences. When the tumor average volume was up to 30 mm³ (about 9 days post-injection), the mice were intravenously (i.v.) injected with the complexes, PBS or empty liposomes; and the mice were intravenously (i.v.) injected with the complexes, PBS or empty liposomes again at 11 days post-injection. In addition, when the tumor average volume was up to 2500 mm³, the mice were sacrificed.

The complexes used in the present example comprised complexes of LPPC and RBDV-IgG1 Fc proteins (LPPC/RBDV protein), complexes of LPPC and IgG1 Fc proteins (LPPC/IgG1 protein), complexes of LPPC, pAAV-MCS/RBDV-IgG1 Fc and RBDV-IgG1 Fc proteins (LPPC/RBDV plasmid/RBDV protein), and complexes of LPPC, pAAV-MCS/IgG1 Fc and RBDV-IgG1 Fc proteins (LPPC/IgG1 plasmid/RBDV protein).

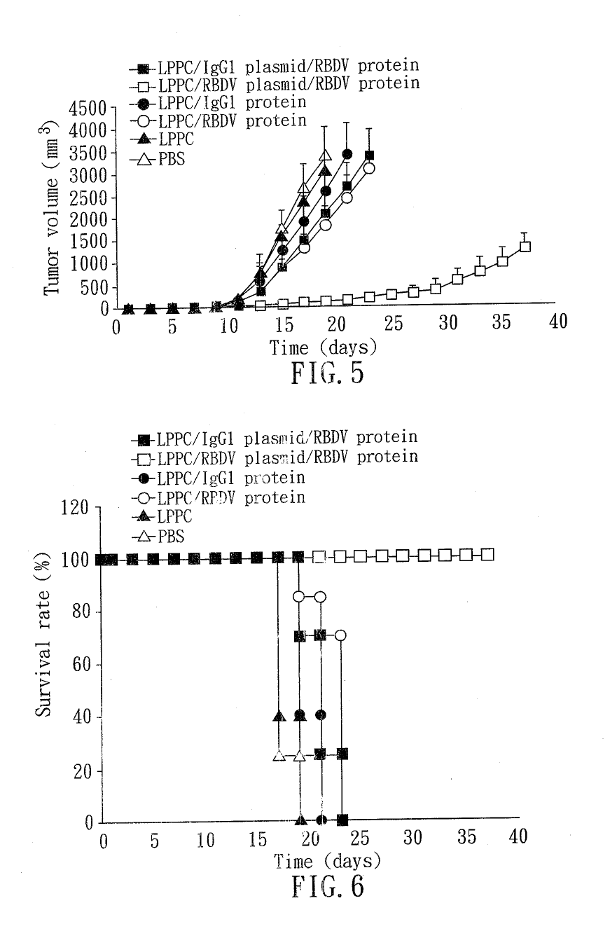

The results of the present example are shown in FIG. 5 and FIG. 6. As shown in FIG. 5, only the complexes containing RBDV plasmids (i.e. LPPC/RBDV plasmid/RBDV protein) can inhibit the tumor growth. In addition, as shown in FIG. 5, only the mice those injected with complexes containing RBDV plasmids (i.e. LPPC/RBDV plasmid/RBDV protein) had survival rate of 100% within 40 days.

Hence, from the results shown in FIG. 5 and FIG. 6, the inhibition effect on the tumor growth as well as the survival rate of mice can be increased when RBDV-IgG1 proteins were expressed in vivo.

In conclusion, RBDV-IgG1 proteins contained in the complexes of RBDV-IgG1 proteins and liposomes can target to tumor cells, and the liposomes contained therein can carry DNA or plasmids capable of expressing RBDV proteins. Hence, when RBDV-IgG1 proteins and DNA (or plasmids) capable of expressing RBDV proteins are prepared with drug carriers such as liposomes to form a pharmaceutical composition, the purpose of inhibiting angiogenesis and tumor growth can be obtained through in vivo expressing RBDV proteins, and the survival rate can further be improved.

Although the present invention has been explained in relation to its preferred embodiment, it is to be understood that many other possible modifications and variations can be made without departing from the spirit and scope of the invention as hereinafter claimed.

What is claimed is:

1. A pharmaceutical carrier for inhibiting angiogenesis, comprising:
a drug carrier; and
a polypeptide linked to a surface of the drug carrier, wherein the polypeptide comprises a receptor binding domain of vascular endothelial growth factor.

2. The pharmaceutical carrier as claimed in claim 1, wherein the receptor binding domain of vascular endothelial growth factor is a receptor binding domain of vascular endothelial growth factor A.

3. The pharmaceutical carrier as claimed in claim 1, wherein the polypeptide further comprises a fragment of immunoglobulin, and the fragment of immunoglobulin is linked to the receptor binding domain of vascular endothelial growth factor.

4. The pharmaceutical carrier as claimed in claim 3, wherein the fragment of immunoglobulin is a constant region fragment of immunoglobulin G1.

5. The pharmaceutical carrier as claimed in claim 1, further comprising a nucleic acid molecule linked to the surface of the drug carrier, wherein the nucleic acid molecule comprises a nucleotide sequence of a receptor binding domain of vascular endothelial growth factor.

6. The pharmaceutical carrier as claimed in claim 5, wherein the nucleic acid molecule is a plasmid containing the nucleotide sequence of the receptor binding domain of vascular endothelial growth factor.

7. The pharmaceutical carrier as claimed in claim 6, wherein the nucleic acid molecule is a plasmid containing the nucleotide sequence of the receptor binding domain of vascular endothelial growth factor and a nucleotide sequence of a fragment of immunoglobulin.

8. The pharmaceutical carrier as claimed in claim 7, wherein the fragment of immunoglobulin is a constant region fragment of immunoglobulin G1.

9. The pharmaceutical carrier as claimed in claim 1, wherein the polypeptide is linked to the surface of the drug carrier by absorption.

10. The pharmaceutical carrier as claimed in claim 5, wherein the nucleic acid molecule is linked to the surface of the drug carrier by absorption.

11. The pharmaceutical carrier as claimed in claim 1, wherein the drug carrier is at least one selected from the group consisting of a liposome, a micelle, a microsphere, a nanoparticle, and a dendrimer.

12. A pharmaceutical composition for inhibiting angiogenesis, comprising:
a pharmaceutical carrier comprising: a drug carrier; and a polypeptide linked to a surface of the drug carrier, wherein the polypeptide comprises a receptor binding domain of vascular endothelial growth factor; and
an active ingredient encapsulated in the pharmaceutical carrier.

13. The pharmaceutical composition as claimed in claim 12, wherein the receptor binding domain of vascular endothelial growth factor is a receptor binding domain of vascular endothelial growth factor A.

14. The pharmaceutical composition as claimed in claim 12, wherein the polypeptide further comprises a fragment of immunoglobulin, and the fragment of immunoglobulin is linked to the receptor binding domain of vascular endothelial growth factor.

15. The pharmaceutical composition as claimed in claim 14, wherein the fragment of immunoglobulin is a constant region fragment of immunoglobulin G1.

16. The pharmaceutical composition as claimed in claim 12, wherein the pharmaceutical carrier further comprises: a nucleic acid molecule linked to the surface of the drug carrier, and the nucleic acid molecule comprises a nucleotide sequence of a receptor binding domain of vascular endothelial growth factor.

17. The pharmaceutical composition as claimed in claim 16, wherein the nucleic acid molecule is a plasmid containing the nucleotide sequence of the receptor binding domain of vascular endothelial growth factor.

18. The pharmaceutical composition as claimed in claim 17, wherein the nucleic acid molecule is a plasmid containing the nucleotide sequence of the receptor binding domain of vascular endothelial growth factor and a nucleotide sequence of a fragment of immunoglobulin.

19. The pharmaceutical composition as claimed in claim 18, wherein the fragment of immunoglobulin is a constant region fragment of immunoglobulin G1.

20. The pharmaceutical composition as claimed in claim 16, wherein the polypeptide is linked to the surface of the drug carrier by absorption.

21. The pharmaceutical composition as claimed in claim 12, wherein the active ingredient is an anti-cancer drug or a nucleic acid molecule, wherein the nucleic acid molecule comprises a nucleotide sequence of a receptor binding domain of vascular endothelial growth factor.

22. The pharmaceutical composition as claimed in claim 12, wherein the drug carrier is at least one selected from the group consisting of a liposome, a micelle, a microsphere, a nanoparticle, and a dendrimer.


SEQUENCE LISTING (8 sequences)

SEQ ID NO: 1 - protein, 352 aa, Artificial
  fusion protein containing the receptor binding domain of human VEGF-A and the constant region fragment of human IgG1
MNFLLSWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIISSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDEGPHHHHHH

SEQ ID NO: 2 - DNA, 5700 bp, Artificial
  cloned plasmid containing the receptor bindiing domain of human VEGF-A and the constant region fragment of human IgG1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcatctcgagtgagcccaaatcttgtgacaaaactcacacatgcccaccgtgcccagcacccgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaggggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagtcgacgagggcccgcatcatcaccatcaccattgaaagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 3 - DNA, 24 bp, Artificial
  synthesized primer
tggtgagagatctggttcccgaaa

SEQ ID NO: 4 - DNA, 24 bp, Artificial
  synthesized primer
tttcgggaaccagatctctcacca

SEQ ID NO: 5 - DNA, 28 bp, Artificial
  synthesized primer
aggatccatgaactttctgctgtcttgg

SEQ ID NO: 6 - DNA, 31 bp, Artificial
  synthesized primer
actcgagttagatccgcataatctgcatggt

SEQ ID NO: 7 - DNA, 24 bp, Artificial
  synthesized primer
cgcatcatcaccatcaccattgaa

SEQ ID NO: 8 - DNA, 32 bp, Artificial
  synthesized primer
agctttcaatggtgatggtgatgatgcgggcc